(19) United States
(12) Patent Application Publication
(10) Pub. No.: US 2025/0243241 A1
(43) Pub. Date: Jul. 31, 2025

(54) MULTI-COMPONENT BUFFER SYSTEM FOR PURIFICATION OF ANTIBODIES

(71) Applicant: INTAS PHARMACEUTICALS LTD., Ahmedabad, Gujarat (IN)

(72) Inventors: Laxmi ADHIKARY, Ahmedabad (IN); Tushar JOGLEKAR, Ahmedabad (IN); Anoop VASUDEVAN, Ahmedabad (IN); Dhrumil MODI, Ahmedabad (IN); Pragati PRAJAPATI, Ahmedabad (IN); Niyati PATEL, Ahmedabad (IN); Mukesh DAKUA, Ahmedabad (IN)

(21) Appl. No.: 18/853,962

(22) PCT Filed: Mar. 23, 2023
(86) PCT No.: PCT/IB2023/052850

§ 371 Date: Oct. 3, 2024

(30) Foreign Application Priority Data
Apr. 4, 2022 (IN) 202221020292

(51) Int. Cl. C07K 1/18; C07K 1/34; C07K 16/28; C07K 16/32

(52) U.S. Cl. CPC C07K 1/18; C07K 1/34; C07K 16/2818; C07K 16/2839; C07K 16/2896; C07K 16/32; C07K 2317/24

(57) ABSTRACT

The present invention relates to multi-component buffer system for purification of Antibodies. Specifically, present invention relates cation exchange chromatography process comprising multi-component buffer system and pH based elution for purification of antibodies. The present invention significantly reduces HMW and LMW species including 2H1L species.

### RELATED APPLICATIONS

This application is related to Indian Provisional Application IN202221020292 filed 4 Apr. 2022 and is incorporated herein in its entirety

### FIELD OF THE INVENTION

The present invention relates to multi-component buffer system for purification of Antibodies. Specifically, present invention relates cation exchange chromatography process comprising multi-component buffer system and pH based elution for purification of antibodies.

### BACKGROUND OF THE INVENTION

Monoclonal antibodies (mAbs) have been successfully employed to target a wide range of therapeutic areas over the last two decades. While mAbs possess a conserved covalent heterotetrameric structure consisting of two disulfide-linked heavy chains, each covalently linked through a disulfide bond to a light chain, these proteins often contain low levels of product-related impurities even after extensive purification steps. Low molecular weight (LMW) species and high molecular weight (HMW) species are both examples of product-related impurities that contribute to the size heterogeneity of mAb products. The formation of HMW species within a therapeutic mAb drug product as a result of protein aggregation can potentially compromise both drug efficacy and safety (e.g. eliciting unwanted immunogenic response). LMW species of any therapeutic protein may result from host cell protease activity during production. LMW species often have low or substantially reduced activity relative to the monomeric form of the antibody, while exposing novel epitopes that can lead to immunogenicity or potentially impact pharmacokinetic properties in vivo. As a result, both HMW and LMW species are considered critical quality attributes that are routinely monitored during drug development and as part of release testing of purified drug substance during manufacturing.

Pertuzumab is a full-length recombinant humanized IgG1(κ) monoclonal antibody (mAb) containing an N-linked oligosaccharide. Pertuzumab is comprised of two heavy chains (448 or 449 amino acid residues, dependent on the presence of a C-terminal lysine) and two light chains (214 amino acid residues) and have a molecular weight of approximately 148,000 Da. Pertuzumab is a HER2 receptor antagonist used for treatment of HER2 positive breast cancer.

Pertuzumab targets the extracellular dimerization domain (Subdomain II) of the human epidermal growth factor receptor 2 protein (HER2) and, thereby, blocks ligand-dependent heterodimerization of HER2 with other HER family members, including EGFR, HER3, and HER4. As a result, Pertuzumab inhibits ligand-initiated intracellular signaling through two major signal pathways, mitogen-activated protein (MAP) kinase, and phosphoinositide 3-kinase (PI3K). Inhibition of these signalling pathways can result in cell growth arrest and apoptosis, respectively. In addition, Pertuzumab mediates antibody-dependent cell-mediated cytotoxicity (ADCC).

The principal chromatographic step used for separation of size variants, i.e., HMW and LMW, from the monomer is cation exchange chromatography. The general chromatographic buffer conditions using single buffering system and salt-based elution could effectively separate HMW but was not effective in resolving LMW species such as 2 heavy 1 light chain (2H1L) fragment.

Therefore, there is desire to provide antibody purification method which separates HMW, LMW species and charge variants consistently.

### OBJECTS OF THE INVENTION

The main object of the present invention is to provide a method for purification of an antibody comprising cation exchange chromatography having multicomponent buffer system.

Another object of the present invention is to provide a method for purification of an antibody, comprising cation exchange chromatography having sodium citrate, sodium phosphate and TRIS as a multicomponent buffer system.

Another object of the present invention is to provide a method for purification of an antibody with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nanofiltration; and
f) Tangential flow filtration

Another object of the present invention is to provide a method for purification of Pertuzumab with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nanofiltration; and
f) Tangential flow filtration

Another object of the present invention is to provide a method for purification of Vedolizumab with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nanofiltration; and
f) Tangential flow filtration

Another object of the present invention is to provide a method for purification of an antibody with cation exchange chromatography, the method comprising steps of:
a) Equilibrating cation exchange column with equilibration buffer comprising about 5-20 mM of sodium citrate, about 5-20 mM of sodium phosphate, and about 5-20 mM of Tris buffer; having pH about 5.0±0.5; and Conductivity of about ≤5 mS/cm followed by loading the protein mixture with binding capacity ≤120 mg/ml of resin;
b) Washing the column with wash buffer comprising about 5-20 mM of sodium citrate, about 5-20 mM of sodium phosphate, and about 5-20 mM of Tris buffer; having pH about 5.0±0.5; and Conductivity of about ≤5 mS/cm; and
c) Eluting the antibody with elution buffer comprising about 5-20 mM of sodium citrate, about 5-20 mM of sodium phosphate, and about 5-20 mM of Tris buffer; having pH about 9.0±1.0; and Conductivity of about ≤10 mS/cm;
   Wherein the antibody is selected from the group consisting of Pertuzumab, Vedolizumab, Daratumumab, and Pembrolizumab.

Another object of the present invention is to provide a method for purification of an antibody with cation exchange chromatography, the method comprising steps of:
a) Equilibrating cation exchange column with equilibration buffer comprising about 10 mM of sodium citrate, about 10 mM of sodium phosphate, and about 10 mM of Tris buffer; having pH about 5.0±0.2; and Conductivity of about 3.0±1.0 mS/cm followed by loading the protein mixture with binding capacity ≤120 mg/ml of resin;
b) Washing the column with wash buffer comprising about 10 mM of sodium citrate, about 10 mM of sodium phosphate, and about 10 mM of Tris buffer; having pH about 5.0±0.2; and Conductivity of about 3.0±1.0 mS/cm; and
c) Eluting the antibody with elution buffer comprising about 10 mM of sodium citrate, about 10 mM of sodium phosphate, and about 10 mM of Tris buffer; having pH about 9.0±0.2; and Conductivity of about 4.0±1.0 mS/cm;
   Wherein the antibody is selected from the group consisting of Pertuzumab, Vedolizumab, Daratumumab, and Pembrolizumab.

In another object of the present invention cation exchange chromatography comprising multicomponent buffer system separates LMW, HMW species and charge variants.

### SUMMARY OF THE INVENTION

The main aspect of the present invention is to provide a method for purification of an antibody comprising cation exchange chromatography having multicomponent buffer system.

Another aspect of the present invention is to provide a method for purification of an antibody, comprising cation exchange chromatography having sodium citrate, sodium phosphate and TRIS as a multicomponent buffer system.

Another aspect of the present invention is to provide a method for purification of an antibody with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nanofiltration; and
f) Tangential flow filtration

Another aspect of the present invention is to provide a method for purification of Pertuzumab with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nanofiltration; and
f) Tangential flow filtration

Another aspect of the present invention is to provide a method for purification of Vedolizumab with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nanofiltration; and
f) Tangential flow filtration

Another aspect of the present invention is to provide a method for purification of an antibody with cation exchange chromatography, the method comprising steps of:
a) Equilibrating cation exchange column with equilibration buffer comprising about 5-20 mM of sodium citrate, about 5-20 mM of sodium phosphate, and about 5-20 mM of Tris buffer; having pH about 5.0±0.5; and Conductivity of about ≤5 mS/cm followed by loading the protein mixture with binding capacity ≤120 mg/ml of resin;
b) Washing the column with wash buffer comprising about 5-20 mM of sodium citrate, about 5-20 mM of sodium phosphate, and about 5-20 mM of Tris buffer; having pH about 5.0±0.5; and Conductivity of about ≤5 mS/cm; and
c) Eluting the antibody with elution buffer comprising about 5-20 mM of sodium citrate, about 5-20 mM of sodium phosphate, and about 5-20 mM of Tris buffer; having pH about 9.0±1.0; and Conductivity of about ≤10 mS/cm;
   Wherein the antibody is selected from the group consisting of Pertuzumab, Vedolizumab, Daratumumab, and Pembrolizumab.

Another aspect of the present invention is to provide a method for purification of an antibody with cation exchange chromatography, the method comprising steps of:
a) Equilibrating cation exchange column with equilibration buffer comprising about 10 mM of sodium citrate, about 10 mM of sodium phosphate, and about 10 mM of Tris buffer; having pH about 5.0±0.2; and Conductivity of about 3.0±1.0 mS/cm followed by loading the protein mixture with binding capacity ≤120 mg/ml of resin;
b) Washing the column with wash buffer comprising about 10 mM of sodium citrate, about 10 mM of sodium phosphate, and about 10 mM of Tris buffer; having pH about 5.0±0.2; and Conductivity of about 3.0±1.0 mS/cm; and
c) Eluting the antibody with elution buffer comprising about 10 mM of sodium citrate, about 10 mM of sodium phosphate, and about 10 mM of Tris buffer; having pH about 9.0±0.2; and Conductivity of about 4.0±1.0 mS/cm;
   Wherein the antibody is selected from the group consisting of Pertuzumab, Vedolizumab, Daratumumab, and Pembrolizumab.

In another aspect of the present invention cation exchange chromatography comprising multicomponent buffer system separates LMW, HMW species and charge variants.

### BRIEF DESCRIPTION OF DRAWINGS

In order that disclosure may be readily understood and put into practical effect, reference will now be made to exemplary embodiments as illustrated with reference to the accompanying FIGURES. The FIGURE with a detailed description below, are incorporated in and form part of the specification, and serve to further illustrate the embodiments and explain various principles and advantages, in accordance with the present disclosure wherein:

FIG. 1: Process Flow of Cation Exchange Chromatography

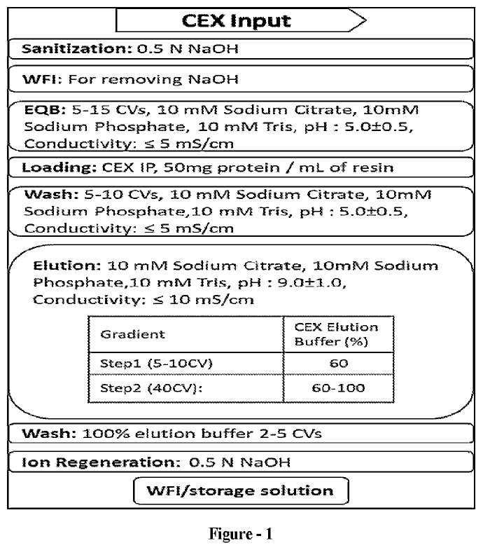

### DETAILED DESCRIPTION OF THE INVENTION

The following is a detailed description of embodiments of the invention. The embodiments are in such details as to clearly communicate the invention. However, the amount of details offered is not intended to limit the anticipated variations of embodiments; on the contrary, the intention is to cover all modifications, equivalents of embodiments, and alternative falling within the spirit and scope of the present invention.

### Definition

The following definitions are provided to facilitate understanding of certain terms used throughout the specification.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The term “about” as used in the present patent specification is meant to specify that the specific value provided may vary to a certain extent.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

The term “low molecular weight (LMW) species” includes but is not limited to precursors, degradation products, truncated species, proteolytic fragments including Fab fragments, Fc or heavy chain fragments, ligand or receptor fragments, 2H1L (2 heavy chains and 1 light chain), H2 (2 heavy chains), HL (1 heavy chain and 1 light chain), HC (1 heavy chain), and LC (1 light chain) species. A LMW species can be any variant which is an incomplete version of the protein product, such as one or more components of a multimeric protein.

“Cation exchange resins” refers to an ion exchange resin with covalently bound negatively charged ligands, and which thus has free cations for exchange with cations in a solution with which the resin is contacted. A wide variety of cation exchange resins are known in the art, for example, those wherein the covalently bound groups are carboxylate or sulfonate.

The term “antibody” herein is used in the broadest sense and specifically covers full length monoclonal antibodies, polyclonal antibodies, multispecific antibodies (e.g. bispecific antibodies) formed from at least two full length antibodies, and antibody fragments, so long as they exhibit the desired biological activity.

An “antibody fragment” comprises a portion of a full length antibody, in particular comprises the antigen-binding or variable region thereof. Examples of antibody fragments include Fab, Fab′, F(ab′)2, and Fv fragments; diabodies; linear antibodies; single-chain antibody molecules; and multispecific antibodies formed from antibody fragment(s).

A “full length antibody” is one which comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variants thereof. In particular the full length antibody has one or more effector functions.

Exemplary antibodies which can be formulated according to the present invention include, but are not limited to the following: Trastuzumab or Pertuzumab or Rituximab or Bevacizumab or Adalimumab or Tocilizumab or Vedolizumab or Denosumab or Ranibizumab or Aflibercept or Pembrolizumab or Daratumumab or Romiplostim or Etanercept or Nivolumab or Atezolizumab or Durvalumab or Guselkumab or Ixekizumab or Secukinumab or Dupilumab or Ocrelizumab, etc.

The main embodiment of the present invention is to provide a method for purification of an antibody comprising cation exchange chromatography having multicomponent buffer system.

Another embodiment of the present invention is to provide a method for purification of an antibody, comprising cation exchange chromatography having sodium citrate, sodium phosphate and TRIS as a multicomponent buffer system.

Another embodiment of the present invention is to provide a method for purification of an antibody with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nanofiltration; and
f) Tangential flow filtration

Another embodiment of the present invention is to provide a method for purification of Pertuzumab with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nanofiltration; and
f) Tangential flow filtration

Another embodiment of the present invention is to provide a method for purification of Vedolizumab with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nanofiltration; and
f) Tangential flow filtration

Another embodiment of the present invention is to provide a method for purification of an antibody with cation exchange chromatography, the method comprising steps of:
a) Equilibrating cation exchange column with equilibration buffer comprising about 5-20 mM of sodium citrate, about 5-20 mM of sodium phosphate, and about 5-20 mM of Tris buffer; having pH about 5.0±0.5; and Conductivity of about ≤5 mS/cm followed by loading the protein mixture with binding capacity ≤120 mg/ml of resin;
b) Washing the column with wash buffer comprising about 5-20 mM of sodium citrate, about 5-20 mM of sodium phosphate, and about 5-20 mM of Tris buffer; having pH about 5.0±0.5; and Conductivity of about ≤5 mS/cm; and
c) Eluting the antibody with elution buffer comprising about 5-20 mM of sodium citrate, about 5-20 mM of sodium phosphate, and about 5-20 mM of Tris buffer; having pH about 9.0±1.0; and Conductivity of about ≤10 mS/cm;
   Wherein the antibody is selected from the group consisting of Pertuzumab, Vedolizumab, Daratumumab, and Pembrolizumab.

Another embodiment of the present invention is to provide a method for purification of an antibody with cation exchange chromatography, the method comprising steps of:
a) Equilibrating cation exchange column with equilibration buffer comprising about 10 mM of sodium citrate, about 10 mM of sodium phosphate, and about 10 mM of Tris buffer; having pH about 5.0±0.2; and Conductivity of about 3.0±1.0 mS/cm followed by loading the protein mixture with binding capacity ≤120 mg/ml of resin;
b) Washing the column with wash buffer comprising about 10 mM of sodium citrate, about 10 mM of sodium phosphate, and about 10 mM of Tris buffer; having pH about 5.0±0.2; and Conductivity of about 3.0±1.0 mS/cm; and
c) Eluting the antibody with elution buffer comprising about 10 mM of sodium citrate, about 10 mM of sodium phosphate, and about 10 mM of Tris buffer; having pH about 9.0±0.2; and Conductivity of about 4.0±1.0 mS/cm;
   Wherein the antibody is selected from the group consisting of Pertuzumab, Vedolizumab, Daratumumab, and Pembrolizumab.

In another embodiment of the present invention the cation exchange chromatographic elution is carried out by multicomponent buffer system with pH based elution.

In another embodiment of the present invention cation exchange chromatography comprising multicomponent buffer system separates LMW, HMW species and charge variants.

The embodiments of the present invention are further described using specific examples herein after. The examples are provided for better understanding of certain embodiments of the invention and not, in any manner, to limit the scope thereof. Possible modifications and equivalents apparent to those skilled in the art using the teachings of the present description and the general art in the field of the invention shall also from the part of this specification and are intended to be included within the scope of it.

### EXAMPLES

### Example 1: Purification of Antibody with Cation Exchange Chromatography Using Multi-Component Buffer System

The stationary phase media used in cation exchange step was cross-linked agarose with the SO3-ligand. The cation exchange column was first equilibrated with 5-15 CVs of equilibration buffer 10 mM sodium citrate, 10 mM sodium phosphate, 10 mM Tris (pH: 5.0±0.5; conductivity: ≤5 mS/cm) followed by loading of the protein mixture with binding capacity ≤120 mg/ml of resin. The loaded protein at cation exchange stage gets bound to the column and washed with 5-10 CVs of equilibration buffer 10 mM sodium citrate, 10 mM sodium phosphate, 10 mM Tris (pH: 5.0±0.5; conductivity: ≤5 mS/cm). After washing, the protein of interest was eluted using 5-50 CVs of elution buffer 10 mM sodium citrate, 10 mM sodium phosphate, 10 mM Tris (pH: 9.0±1.0; conductivity: ≤10 mS/cm). The elution was carried out in two stages: 40% to 60% elution buffer in 5-10 CVs and 60% to 100% elution buffer in about 40 CVs.

With the above chromatographic conditions that used multiple buffer components and a pH-based elution instead of a single buffer component and salt-based elution, the LMWs were effectively separated to achieve higher monomer content thereby ensuring better safety and efficacy of the drug.

TABLE 1
Comparison of single buffer and multi-
buffer chromatography system

| Process | Batch No. | LMW (%) | 2H1L (%) |
|---|---|---|---|
| Single buffer | 1 | 2.3 | 1.6 |
| | 2 | 4.1 | 2.7 |
| Multi-buffer | 1 | 1.5 | 1.0 |
| (Present Invention) | 2 | 1.6 | 1.0 |

The table-1 shows the levels of LMW and specifically 2H1L species for Pertuzumab with the traditional single buffer process and the novel multi-buffer process. As it can be seen that there was significant decrease in the LMW (%) and 2H1L (%) species with use of multi buffer system of the present invention.

To further confirm the above result multiple batches of antibody was purified using above described process of the present invention (multi-component buffer system) and conventional single buffer process. Following table-2 and table-3 shows result obtained using said conventional process and process of the present invention respectively.

TABLE 2
Conventional Cation Exchange Chromatography
(single component buffer)

| | % | | | | | |
|---|---|---|---|---|---|---|
| Sample | Acidic | % | % Basic | | Total % | % Main |
| Name | variants | Purity | variants | %2H1L | LMW | peak |
| P34-BM-Engg- | 18.63 | 72.54 | 8.83 | 0.47 | 2.20 | 97.34 |
| 02 CEX Input | | | | | | |
| P34-BM-Engg- | 19.09 | 75.95 | 4.96 | 0.52 | 2.14 | 97.35 |
| 02 CEX Output | | | | | | |

TABLE 3
Present Invention Cation Exchange Chromatography
(Multi Component Buffer)

| | % | | | | | |
|---|---|---|---|---|---|---|
| Sample | Acidic | % | % Basic | | Total % | % Main |
| Name | variants | Purity | variants | %2H1L | LMW | peak |
| P34-BM-0001 | 17.27 | 73.87 | 8.86 | 4.6 | 7.6 | 92.4 |
| CEX Input | | | | | | |
| P34-BM-0001 | 13.03 | 77.51 | 9.45 | 2.9 | 4.3 | 95.7 |
| CEX Output | | | | | | |

As evident from the above table, %21HL (P34-BM-0001 CEX Input: 4.6% and P34-BM-0001 CEX output: 2.9%) was ^{˜}63% less & Total % LMW (P34-BM-ENGG-05 CEX input: 7.6% and P34-BM-ENGG-05 CEX output: 4.3%) was ^{˜}56% less when the cation exchange chromatography with multi-component buffer & pH-based elution was used. Whereas, in the conventional process there was no significant change in % basic variants, % 2H1L and total % LMW.

1. A method for purification of an antibody comprising cation exchange chromatography having multicomponent buffer system.

2. The method for purification of an antibody according to claim 1, wherein said multicomponent buffer system comprises sodium citrate, sodium phosphate and TRIS.

3. A method for purification of an antibody with reduced level of size variants, comprising:
a) Protein A chromatography;
b) Low pH treatment and depth filtration;
c) Anion exchange chromatography;
d) Cation exchange chromatography having multicomponent buffer system, wherein multicomponent buffer system comprises sodium citrate, sodium phosphate and TRIS;
e) Nano filtration; and
f) Tangential flow filtration.

4. The method for purification of an antibody according to claim 3, wherein multicomponent buffer system comprises comprising about 5-20 mM of sodium citrate, about 5-20 mM of sodium phosphate, and about 5-20 mM of Tris.

5. A method for purification of an antibody with cation exchange chromatography, the method comprising steps of:
a) Equilibrating cation exchange column with equilibration buffer comprising about 5-20 mM of sodium citrate, about 5-20 mM of sodium phosphate, and about 5-20 mM of Tris buffer; having pH about 5.0±0.5; and Conductivity of about ≤5 mS/cm followed by loading the protein mixture with binding capacity ≤120 mg/ml of resin;
b) Washing the column with wash buffer comprising about 5-20 mM of sodium citrate, about 5-20 mM of sodium phosphate, and about 5-20 mM of Tris buffer; having pH about 5.0±0.5; and Conductivity of about ≤5 mS/cm; and
c) Eluting the antibody with elution buffer comprising about 5-20 mM of sodium citrate, about 5-20 mM of sodium phosphate, and about 5-20 mM of Tris buffer; having pH about 9.0±1.0; and Conductivity of about ≤10 mS/cm.

6. The method of purification of antibody according to claim 5, wherein the method comprising steps of:
a) Equilibrating cation exchange column with equilibration buffer comprising about 10 mM of sodium citrate, about 10 mM of sodium phosphate, and about 10 mM of Tris buffer; having pH about 5.0±0.2; and Conductivity of about 3.0±1.0 mS/cm followed by loading the protein mixture with binding capacity ≤120 mg/ml of resin;
b) Washing the column with wash buffer comprising about 10 mM of sodium citrate, about 10 mM of sodium phosphate, and about 10 mM of Tris buffer; having pH about 5.0±0.2; and Conductivity of about 3.0±1.0 mS/cm; and
c) Eluting the antibody with elution buffer comprising about 10 mM of sodium citrate, about 10 mM of sodium phosphate, and about 10 mM of Tris buffer; having pH about 9.0±0.2; and Conductivity of about 4.0±1.0 mS/cm.

7. The method of purification of antibody according to claim 1, wherein antibody is selected from the group consisting of Pertuzumab, Vedolizumab, Daratumumab, and Pembrolizumab.

8. The method of purification of antibody according to claim 3, wherein antibody is selected from the group consisting of Pertuzumab, Vedolizumab, Daratumumab, and Pembrolizumab.

9. The method of purification of antibody according to claim 5, wherein antibody is selected from the group consisting of Pertuzumab, Vedolizumab, Daratumumab, and Pembrolizumab.
